(19) United States
(12) Patent Application Publication
(10) Pub. No.: US 2008/0051804 A1
(43) Pub. Date: Feb. 28, 2008

(54) Tube, stent and collar insertion device

(76) Inventors: Shayn Peirce Cottler, Charlottesville, VA (US); Bradley W. Kesser, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/903,007

(22) Filed: Sep. 20, 2007

Related U.S. Application Data
(63) Continuation-in-part of application No. PCT/US06/17906, filed on May 5, 2006.
(60) Provisional application No. 60/677,902, filed on May 5, 2005.
(60) Provisional application No. 60/846,072, filed on Sep. 20, 2006.
(60) Provisional application No. 60/850,669, filed on Oct. 10, 2006.

(51) Int. Cl. A61F 11/00

(52) U.S. Cl. 606/109

(57) ABSTRACT

A surgical device and method designed to make insertion of pressure equalization tubes for effusion of the inner ear faster, more reliable, and safer by combining the placement of the pressure equalization tube with suction of the inner ear, decreasing the number of instruments and instrument passes into and out of the ear canal. A surgical insertion tool permits the direct coaxial insertion of the stent through the surgically created ostium, and which may be under direct, clear endoscopic control.

### RELATED APPLICATIONS

This application is a continuation-in-part of PCT International application No. serial No. PCT/US2006/17906, filed May 5, 2006, entitled, “Surgical Tool and Insertion Device for Tube Placement,” which claims benefit of priority from U.S. Provisional Application Ser. No. 60/677,902, filed May 5, 2005, entitled “Surgical Tool and Inserter Device for Tube-Placement Solution During Myringotomy,” of which the disclosures are hereby incorporated by reference herein in their entirety. Additionally, the present patent application claims benefit of priority from U.S. Provisional Application Ser. No. 60/846,072, filed Sep. 20, 2006, entitled “Pressure Equalization (PE) Tube Insertion Device and Collar Suction Adaptation and Related Method,” and U.S. Provisional Application Ser. No. 60/850,669, filed Oct. 10, 2006, entitled “Sinus Stent Insertion Device and Related Method,” of which the disclosures are hereby incorporated by reference herein in their entirety.

### FIELD OF THE INVENTION

The present invention relates generally to surgical devices for ventilation of the inner ear; and/or facilitate insertion of sinus stents and/or suction of the sinus fluids with insertion of the stent.

### BACKGROUND OF THE INVENTION

Pressure Equalization (PE) Tube and Collar Insertion Device

The placement of pressure equalization (PE) tubes into the tympanic membrane (ear drum) is the most commonly performed surgical procedure in the United States, with an occurrence of 1.05 million PE tubes placed per year. PE tubes allow fluid to drain from the middle ear, and, more importantly, ventilate the middle ear to prevent fluid accumulation. Fluid in the middle ear, otitis media with effusion (OME), affects over 2 million individuals each year and is the most common diagnosis made in pediatric offices. Fluid in the middle ear space puts pressure on the tympanic membrane and can cause pain, hearing loss, speech and language delays, and structural damage to the tympanic membrane and other structures vital to the processing of sound information. To relieve this pressure and drain the fluid, a surgeon often makes an incision in the tympanic membrane (myringotomy), aspirates the fluid, and places a PE tube in the membrane incision. The tympanic membrane heals around the tube, and this patent opening between the middle ear and ear canal ventilates the middle ear space.

A traditional insertion method of a PE tube requires three different instruments (alligator forceps, Rosen needle, and suction). The number of instruments and instrument passes through the ear drum takes time and creates risk.

An alternative approach employs a PE tube including a notch adapted for cooperation with a mating key on the insertion device to facilitate alignment of the guide portion of the flange with the incision (See U.S. Pat. No. 4,468,218). However even this approach was unsatisfactory because, but not limited thereto, the proprietary PE tube could only be used at particular locations of the ear drum and at limited angles.

There is therefore a need in the art for an effective surgical device to provide better modes of treatment. Particular needs remain for expediting the procedure and improve patient safety by reducing the total number of instruments that need to be passed through the ear canal during the procedure.

Stent and Collar Insertion Device

Chronic rhinosinusitis (CRS) is the most common chronic ailment in the United States with a prevalence of 134.4 cases per 1,000. 35 million cases are diagnosed each year in the United States, accounting for 11.9 million patient visits per year. The treatment of CRS is initially medical management, but for patients refractory to medical care, surgery is often recommended.

First introduced in 1978, functional endoscopic sinus surgery (FESS) seeks to open the narrowed or obstructed natural drainage pathways of the sinuses. The sinuses are air-filled cavities in the face and skull and are lined by mucus membrane. The mucus membrane makes a blanket of mucus that traps debris and particulate matter that enter the nose or sinuses. The layer of mucus is transported to discrete openings in the sinuses that drain into the nasal cavities. As a result of chronic infection/inflammation, polyps, scarring, allergic disease, or congenital abnormalities, the natural drainage pathways for the sinuses may become obstructed. FESS opens these drainage pathways and preserves the healthy underlying mucus membrane (unless the mucus membrane itself is diseased) so the sinus can drain properly, physiologically.

Any surgery, whether on skin, in the abdomen, or in the nose, causes the formation of scar tissue. Vigorous scar tissue formation in the nose can jeopardize an otherwise successful endoscopic surgical procedure by scarring closed the sinus drainage pathways. To prevent scar tissue formation and to keep patent a surgically created ostium, otolaryngology sinus surgeons often place surgical stents for a period of time. These stents hold open the surgically enlarged drainage pathway and prevent scarring or stenosis (narrowing). Stents are most commonly placed in the frontal recess (drainage pathway for the frontal sinus) and sphenoid ostium (drainage pathway for the sphenoid sinus) as these two surgically enlarged openings have the greatest tendency to stenose.

### SUMMARY OF INVENTION

An aspect an embodiment of the present invention consists of, but not limited thereto, a design providing suction of the middle ear fluids with insertion of a ventilation tube (i.e., a “2-in-1” approach). The ventilation tube may be a PE tube or the like.

Various embodiments of the present invention may pertain directly to, among other things:
Making the insertion of PE tubes (ear tubes) easier, faster, and safer,
Reducing the total number of instruments needed for the procedure by combining suction with PE tube insertion,
Reducing the total number of instruments passes through the ear canal, expediting the procedure and reducing risk to the patient,
Allowing the user a clear line of sight and “feel” of insertion,
Fitting most PE tube model on the market (many different manufacturers),
Creating disposable or reusable forms easily.

An aspect of an embodiment of the present invention of present invention provides a surgical device in communication with a suction source, wherein the device for suctioning of the middle ear with the insertion and placement of a ventilation tube in the tympanic membrane. The device comprising: a tubular rod portion having a longitudinal axis, a proximal end, and a distal end with ventilation tube disposed thereon; an insertion collar portion coaxial to the longitudinal axis of the rod portion to effectively apply a force against the mounted ventilation tube through the tympanic membrane when the rod portion is advanced; and wherein the rod portion is adapted to allow suction of the middle ear through the distal end of the rod portion during the insertion and/or placement of the ventilation tube.

An aspect of an embodiment of the present invention includes a tubular rod attached to a suction source at one end with an insertion collar at the other end to apply pressure against the PE tube when the rod is advanced. By tubular rod, we intend not just circular, but any conduit with any shaped cross section as desired or required.

An aspect of an embodiment of the present invention may include an insertion collar located at a distance from the end greater than the length of the PE tube. In some embodiments, an insertion collar may be located at a distance corresponding to the length of the PE tube. In other embodiments, an insertion collar may be located at a distance less than the length of the PE tube.

An aspect of an embodiment of the present invention may include an insertion collar having an extension portion and a non-extension portion, extension portion having a smaller cross section than non-extension portion, thereby defining a shoulder there between. In other embodiments, at least a portion of a PE tube extends over at least a portion of the extension portion, wherein the applied force is effected by the shoulder contacting the tube.

An aspect of an embodiment the invention may include a PE tube with a portion having a larger cross section to form a shoulder which force is applied against when the rod is advanced. In some embodiments, a portion of the PE tube extends over the at least a portion of the insertion collar.

An aspect of the invention may include a detachable collar which can be installed or removed from a tubular rod.

An aspect of the invention may include an insertion collar and tubular rod which are one integral component. It should be appreciated that any of the elements, structures or portions discussed or taught may be integrally formed with any of one or more elements, structures or portions.

An aspect of the invention may include an insertion collar and tubular rod which are radially axisymmetric.

An aspect of the invention may include an insertion collar made of a clear material to provide light penetration and/or unobstructed view of the ear.

An aspect of the invention may include a blade. In some embodiments the blade is retractable from the tubular rod. In other embodiments, the blade retraction is controllable by the user.

In an aspect, the invention includes a device made of disposable material. In some embodiments the device is provided in a sterile package or container. In other embodiments the device is pre-loaded with a PE tube.

In an aspect, the invention includes a collar incorporating one or more protrusions which apply force to the PE tube when the rod is advanced.

In other aspects, the invention includes a collar extending along the rod, creating a sleeve.

In other aspects, the invention includes a device which is exceptionally streamlined and facilitates a clear line of sight for the surgeon to view the incision in the tympanic membrane without unnecessary obstruction by the device.

In another aspect, the invention includes a device which allows the surgeon to “feel” the insertion of the PE tube by letting the surgeon apply the amount of pressure/force he/she desires when inserting the tube into the incision, thus allowing him/her complete control over the insertion event.

Some aspects of various embodiments of the present invention provides, but not limited thereto, a device and related method to make the insertion of sinus stents easier, faster, and safer. Some aspects of various embodiments of the present invention provides, among other things, suction of the sinus fluids with insertion of the stent.

Some aspects of various embodiments of the present invention provide, but not limited thereto, the surgeon both suction and fingertip control of stent insertion at the point of insertion. By providing suction with an insertion tool, placement of sinus stents becomes, for example, better visualized, more accurate and reliable, and quicker.

With an approach, the surgeon performs the operation under endoscopic guidance, either by viewing a monitor that receives the image in real-time from a camera mounted on the endoscope or by viewing the surgical field through the endoscope itself. In this manner, the surgeon has real-time control of the operation and is able to manipulate instruments inside the nasal cavity.

Some aspects of various embodiments of the present invention provides, but not limited thereto, a suction capability with stent insertion. In some embodiments, the sinus stent is mounted on a hollow stainless steel rod that serves as the suction device. In some embodiments, a trigger mechanism actuates a second, pushing rod over the suction tube to advance the stent and push it into position. Suction through the inner rod is carefully regulated by placing the thumb over a control hole, a commonly used mechanism in the ENT arena for controlling suction. Dimensions are scaled for the commonly used sinus stents and the anatomical constraints/dimensions of the nasal cavity, the field of surgery.

Some exemplary novel characteristics associated with some aspects of various embodiments of the present invention device and method include, but not limited thereto, the following:
Providing suction with stent insertion allows the surgeon to clear the field of mucus, pus, blood, or any other secretions that may impair visibility. The nasal cavity is a tight space in which to operate, and any secretions impair the surgeon's visibility, especially if they splatter on the endoscope. The surgeon's only view inside the nasal cavity is through the endoscope, so it is imperative that the endoscope stay clean and free of splatter.
Providing suction with the insertion tool will allow the surgeon to keep his surgical field and endoscope clear of secretions while giving the surgeon fingertip control of stent insertion.

As such, conventional stents are currently placed by grasping the stent with an alligator forceps and sliding them into position. There is no suction capability with the current conventional technology.

An aspect of various embodiments of the present invention insertion tool permits the direct coaxial insertion of the stent through the surgically created ostium under direct, clear endoscopic control.

An aspect of an embodiment of the present invention provides a surgical device for the insertion and placement of a stent in the sinus ostium. The device comprising: a rod portion having a longitudinal axis, a proximal end, and a distal end with the stent mounted disposed thereon; and an insertion collar portion coaxial to the longitudinal axis of the rod portion to effectively apply a force against the mounted stent through the sinus ostium when the rod portion is advanced.

These and other advantages and features of the invention disclosed herein, will be made more apparent from the description, drawings and claims that follow.

### BRIEF SUMMARY OF THE DRAWINGS

The foregoing and other objects, features and advantages of the present invention, as well as the invention itself, will be more fully understood from the following description of preferred embodiments, when read together with the accompanying drawings, in which:

FIG. 1(A) provides a schematic elevation view of the surgical device.

FIGS. 1(B)-(D) provide enlarged views of various collar designs.

FIGS. 2(A)-(B) provide schematic elevation views of PE tube and insertion collar in preloaded and loaded positions, respectively.

FIGS. 3(A)-(C) provide schematic elevation views of various possible locations for the insertion collar.

FIG. 4 provides a schematic elevation view of insertion collar with an extension portion with cross section smaller than non-extension portion.

FIG. 5 provides a schematic elevation view of PE tube with internal shoulder lumen and corresponding insertion collar configuration.

FIGS. 6(A)-(C) provide schematic elevation views of a rod with: the detachable collar being in pre-installation; the detachable collar being in post-installation and the PE tube being preloaded; and detachable collar being in post-installation and the PE tube being loaded, respectively.

FIG. 7 provides a schematic elevation view of exemplary device.

FIGS. 8(A)-(B) provide a schematic elevation view and top plan view of an incorporation of a myringotomy blade, respectively.

FIG. 9. provides a schematic elevation view of an exemplary nasal stent insertion device.

FIG. 10. provide a schematic elevation view of an exemplary nasal stent insertion device.

### DETAILED DESCRIPTION OF THE INVENTION

An aspect of an embodiment of the invention is, but not limited thereto, providing easier, faster and safer insertion of ventilation tubes, such as a PE tube or the like. Some aspects of the present invention may include, but not limited thereto, as discussed below in the exemplary embodiments.

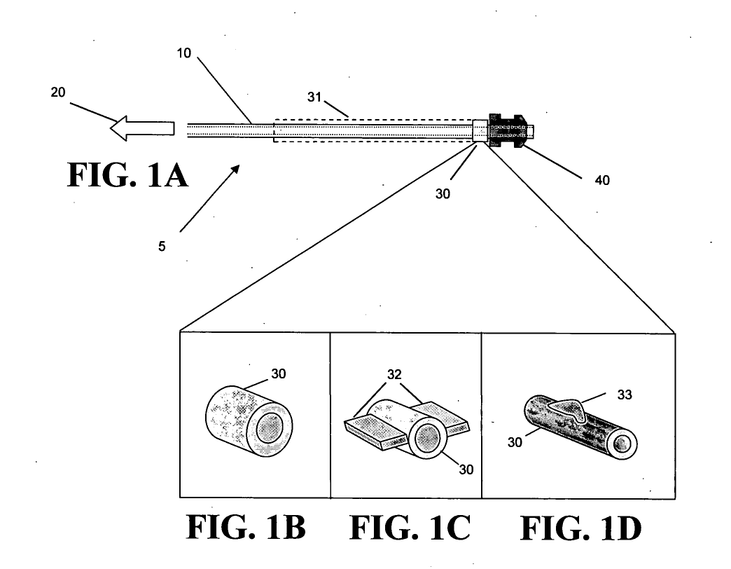

FIG. 1(A) illustrates one embodiment of the invention. The surgical device 5 in communication with a suction source 20, for suctioning of the middle ear with the insertion and placement of a ventilation tube 40, such as a PE tube, or the like, in the tympanic membrane. The device may comprise: a tubular rod 10 having a longitudinal axis, a proximal end, and a distal end with ventilation tube mounted therein; an insertion collar 30 coaxial to the longitudinal axis of the rod to effectively apply a force against the mounted PE tube through the tympanic membrane when the rod is advanced; and wherein the rod is adapted to allow suction of the middle ear through the distal end of the rod during the insertion and/or placement of the PE tube. By tubular rod, we intend not to limit it to just circular, but rather any conduit with any shaped cross section as desired or required. The insertion collar may incorporate one or more protrusions, wherein the applied force is effected by the protrusions contacting the tube.

Some examples of insertion collar designs are shown in FIGS. 1(B)-1(D).

FIG. 1(B) represents a cylindrical collar. FIG. 1(C) represents a collar incorporating wings 32. FIG. 1(D) represents a collar comprising a protrusion 33, for example spot weld bump or the like, or any effective protrusion. As shown by the dotted line extending from the insertion collar, the collar can extend any distance along the rod, forming an extended sleeve 31.

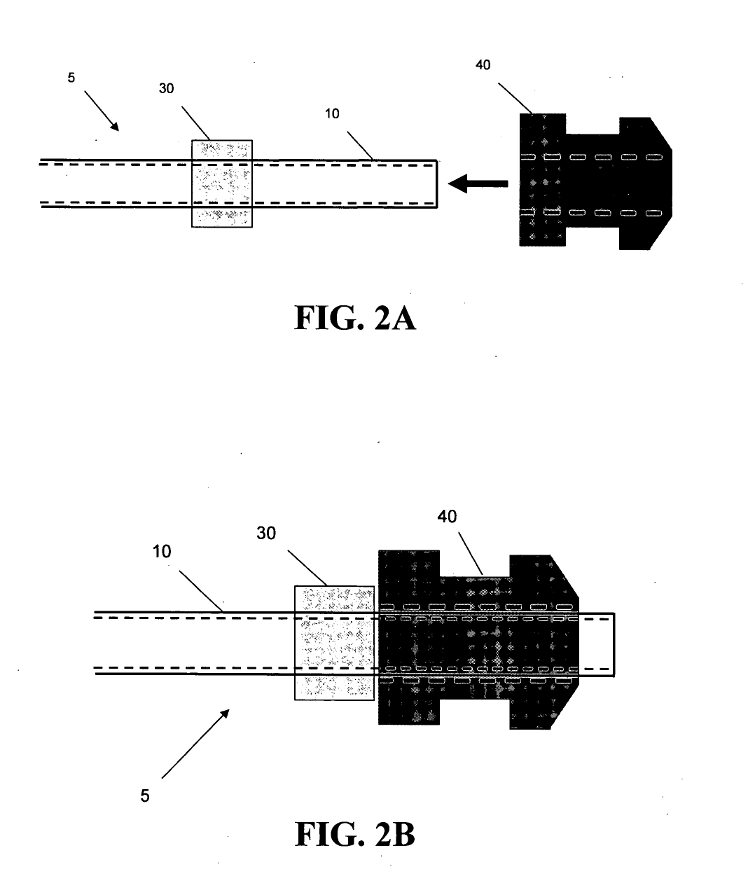

As shown in FIG. 2(A), the PE tube 40 may be initially loaded onto the surgical device 5 by sliding it over a portion of the rod 10 extending beyond the insertion collar 30, towards the insertion collar. FIG. 2(B) shows the PE tube 40 loaded on the rod 10 adjacent to the insertion collar 30.

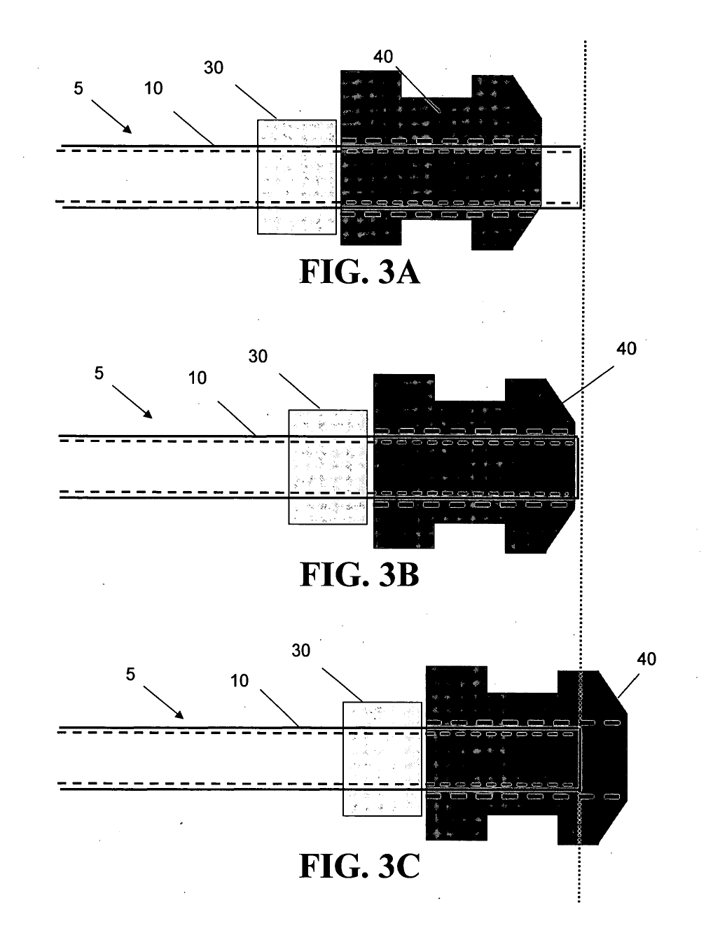

The insertion collar may be located at various distances from the distal end of the rod. FIGS. 3(A)-(C) illustrates a few examples. FIG. 3(A) depicts the insertion collar 30 located at a distance from the distal end of said rod 10 greater than the axial length of said PE tube 40. FIG. 3(B) depicts the insertion collar 30 located at a distance from the distal end of said rod 10 corresponding to the axial length of said PE tube 40. FIG. 3(C) depicts the insertion collar 30 located at a distance from the distal end of said rod 10 less than the axial length of said PE tube 40.

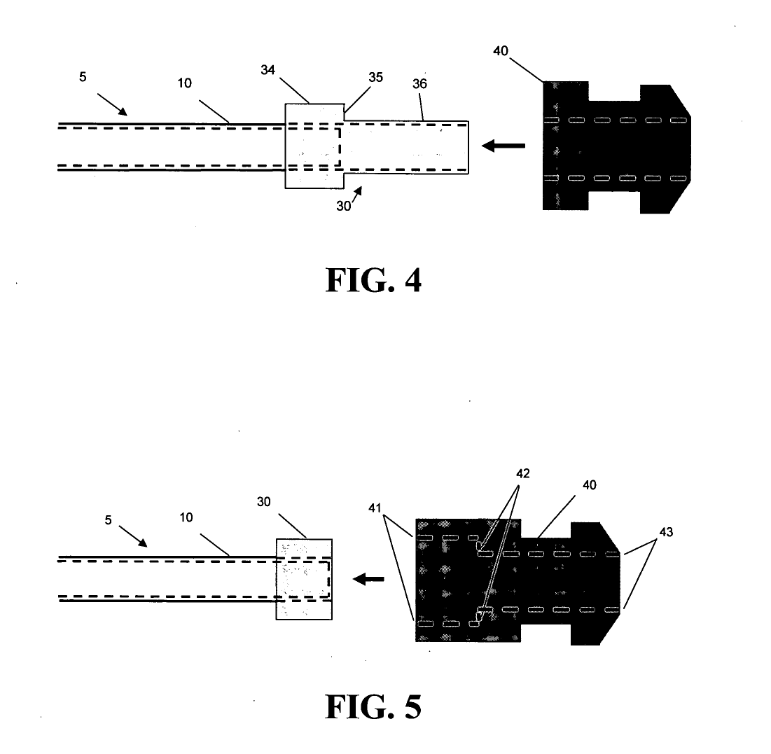

FIG. 4 illustrates how the insertion collar 30 may incorporate an extension portion 36 and a non-extension portion 34. For instance, the extension portion may have a smaller cross section than non-extension portion thereby defining a shoulder 35 there between. At least a portion of the PE tube may extend over at least a portion of the extension portion, wherein the applied force is effected by the shoulder contacting the tube 40.

FIG. 5 illustrates how the PE tube 40 may incorporate a proximal lumen portion 41 and distal lumen portion 43. For instance, the distal lumen portion may have a smaller cross section than proximal lumen portion thereby defining a shoulder lumen 42 there between. At least a portion of the PE tube may extend over at least a portion of the collar 30, wherein the applied force is effected by said shoulder lumen contacting said collar.

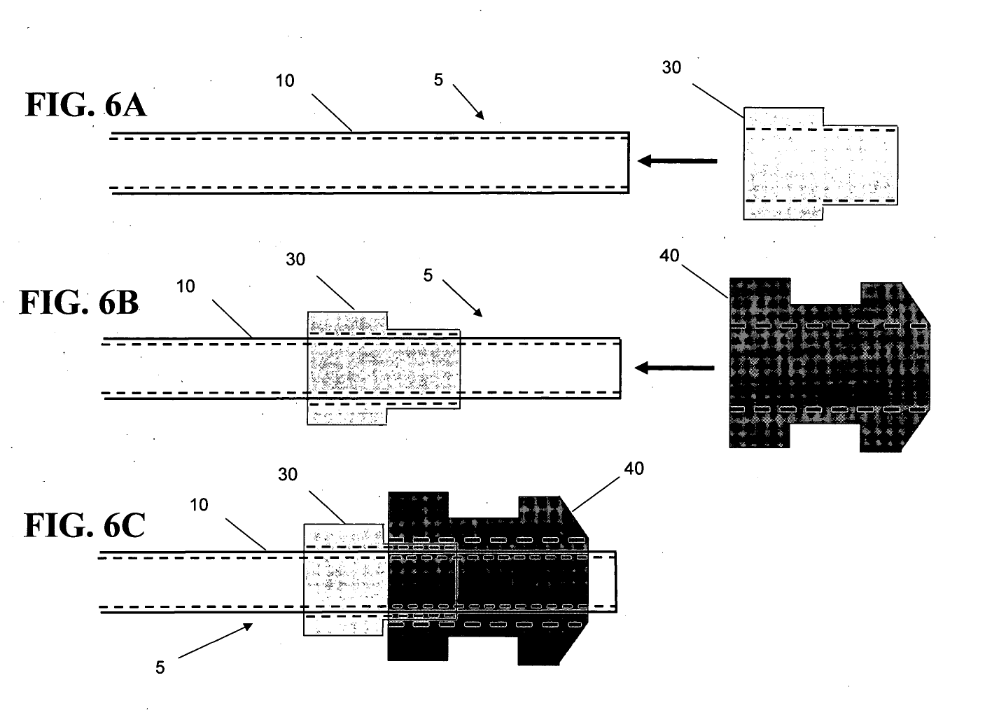

The insertion collar may be one integral piece or detachable (or a combination thereof), wherein it can be installed on/or removed from the tubular rod. FIG. 6(A) depicts an example of a detachable collar 30 being installed on a tubular rod 10. FIG. 6(B) depicts an example of a detachable collar 30 installed on a tubular rod 10 and a PE tube 40 loading onto the surgical device. FIG. 6(C) depicts an example detachable collar 30 installed on a tubular rod 10 with a PE tube 40 loaded on the surgical device 5.

It should be appreciated that with regards to various embodiments of the present invention the rod can extend to the end of the collar, at some location along the collar, but not all the way to the end of the collar, or extend beyond the collar.

Similarly, it should be appreciated that with regards to various embodiments of the present invention the rod can extend to the end of the PE tube, at some location along the PE tube, but not all the way to the end of the PE tube, or extend beyond the PE tube.

The insertion collar and rod may be radially axisymmetric. The insertion collar may be made of a clear material to provide light penetration and/or an unobstructed view of the ear.

The device may also comprise of a blade. The blade may be retractable from the rod. The retraction may be controlled by the user.

The device may be made of disposable material and may be provided in a sterile package or container. The device may be pre-loaded with a PE tube or any components discussed herein.

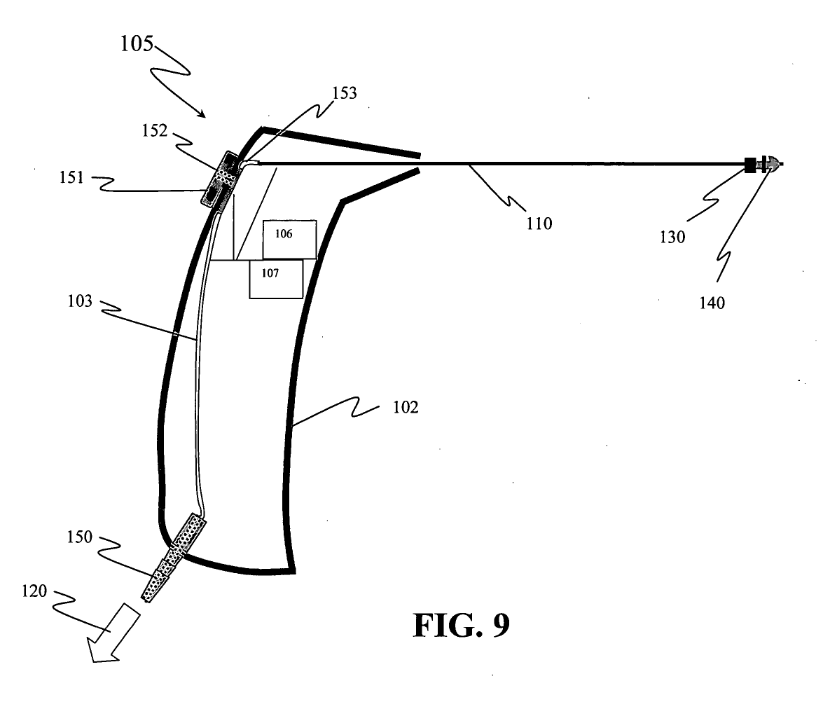

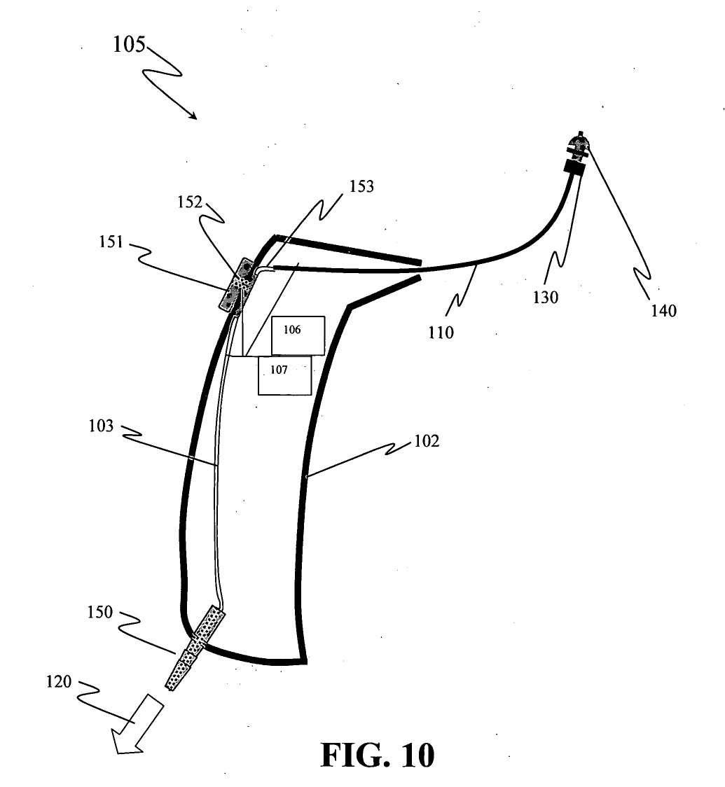

Referring generally to embodiments shown in FIGS. 9 and 10, the sinus stent 140 may be placed on distal end of suction tube 103 or rod 110. The suction tube 103 or rod 110 has an insertion collar 130 to facilitate “pushing” of the stent 140 into place. The collar is affixed over the suction tube allowing the simultaneous insertion of the stent and suctioning of secretions. It should be appreciated that the suction function and structure is not necessarily included in all of the embodiments discussed herein. Further, the casing 102 (or housing) is optional as desired and required.

The suction tube 103 or rod 110 may be a hollow or solid tube (as applicable) that runs back to the thumb control 152 that may be located on the back of the device 105 or as desired or required. The proximal end of the suction connects to wall vacuum source 120, possibly via a suction coupling 150.

When the surgeon's thumb (or user in general) is placed over the suction control pore (hole) 152 in the thumb control 152, this flow outlet is effectively blocked creating a “closed loop”, thus facilitating suction of secretions by the wall vacuum. When the thumb is removed so as to expose this flow outlet, creating a “leak” in the system, suctioning of fluid ceases.

The device enables fingertip stent insertion and thumb-controlled suction simultaneously.

Still referring generally to embodiments shown in FIGS. 9 and 10, the sinus stent 140 may also include a light source 106 and/or image or video capture device 107 such as a camera or the like to assist in operating the device or viewing or recording while operating the device. The light source 106 and/or image or video capture device 107 may be mounted or disposed in, on or outside the device, such as at the rod 110, tube 103, aperture 152, or collar 130 or any combination thereof. The light source 106 and/or image or video capture device 107 may be in communication locally or remotely with the device 140.

Although not illustrated, a pusher tube may be provided adjacent to pusher tube. The pusher tube is attached to a slider. The pusher tube is activated by pulling the trigger, which advances the slider forward in the glide track advancing the pusher tube. A spring returns the slider and pusher tube to the original position, when the trigger is released. The suction tube or rod may be a hollow tube that runs back to the thumb control

Although not illustrated, the surgical device may incorporate a blade which may be retracted by the user. The cutting blade may be mounted on a rod (‘blade rod’). The blade rod may run inside, outside or within the rod or tube for example.

Referring generally to FIG. 10, an embodiment is illustrated wherein the suction tube 103 or rod 110 is bent so as to facilitate easier access through the nose and into the frontal recess.

Still referring generally to embodiments shown in FIGS. 9 and 10, an embodiment may provide a fiber optic light mounted on the nose (or desired location) of the device 105 to facilitate visualization.

An embodiment may provide an endoscopic camera or other tracking/visualizing device mounted on the nose (or desired location) of the device 105 to enable visualization of the procedure.

It should be appreciated that various sizes, dimensions, contours, rigidity, shapes, flexibility and materials may be varied and utilized as desired or required.

It should be appreciated that any of the components, elements, sizes, characteristics, integrations, separateness, disposability, detachability, integration, and functions associated with any of the embodiments (or its components or sub-components) explicitly taught or suggested or inferred may be interchanged, added, removed, augmented, resized, contoured, or replaced with any of the components, elements, sizes and functions associated with other respective embodiments herein. Such components, elements, sizes, characteristics, integrations, separateness, disposability, detachability, integration, and functions may include, but not limited thereto, the following reference numbers: 5, 10, 20, 30, 31, 32, 33, 34, 35, 36, 40, 41, 42, 43, 50, 51, 52, 53, 60, 61, 62, 63, 64, 102, 103, 105, 106, 107, 110, 120, 130, 140, 150, 151, 152, and 153.

Practice of the invention will be still more fully understood from the following examples, which are presented herein for illustration only and should not be construed as limiting the invention in any way.

### EXAMPLE 1

### Reusable Device

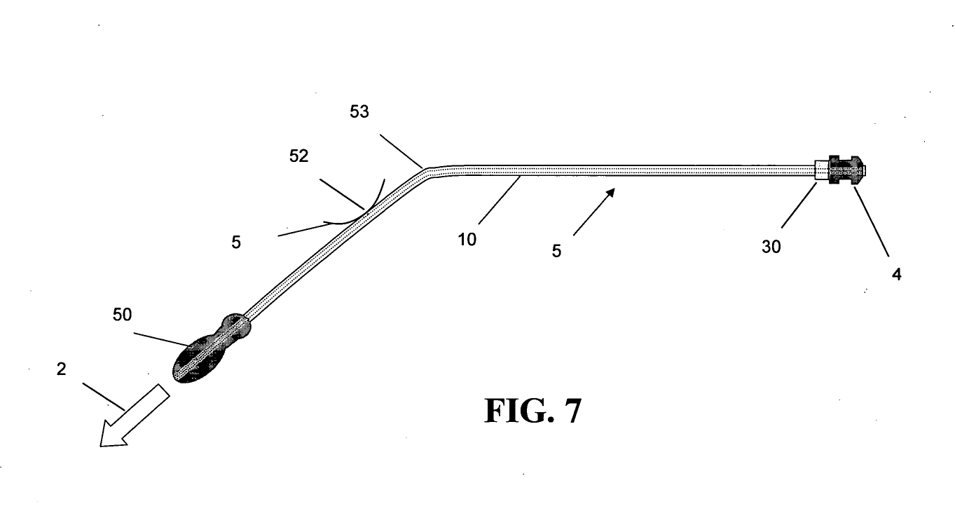

FIG. 7 depicts an exemplary device that may be reusable and made out of stainless steel (or any material as desired), much like the existing suctioning device, so that it can be autoclaved between uses. It should be appreciated that the device may be made of materials that lend itself to be disposable. It may look and feel similar to the existing suction device. It consists of a thin tube 10 (o.d. ranging from 0.86 mm to 1.5 mm, and wall thickness varying accordingly) with a slight angle 53 (approximately 45 deg.) approximately ⅓ of the way from the proximal end of the tube—note that the “proximal end” is defined as the end or region of the tube/device that hooks into the wall suction; the “distal end” is defined as the end or region that bears the ear tube 40 and is inserted into the ear drum. At the proximal end of the tube is a hub connector 50 for a vacuum wall tube.

However, among other things, it contains a collar 30 strategically located near the tip of the device (within 0.5″ of the tip), which effectively pushes the tube into the myringotomy incision. The collar is firmly adhered or press-fit into place so that it is stationary on the outside of the tube. The collar is made out of clear plastic, opaque rubber, or metal. The collar is just large enough to provide adequate pushing force, but small enough to facilitate easy viewing of the ear tube situated at the tip of the device (so as not to obstruct the surgeon's view of the PE tube or the incision). The surgeon controls suction using the existing schema for suction control: a thumb rest 51 containing a hole 52 that is connected to the suction tube over which the surgeon places his/her thumb to permit suction and removes the thumb to “break the circuit” and cease suction. As in the existing suction device, this new device connects to the wall vacuum at its distal end via a standard coupling mechanism.

PE tubes come in all different shapes and sizes, but they can be broadly categorized based on their inner diameters. This device may come in 3 different sizes (or as desired) so as to accommodate all of the PE tubes currently on the market (with the exception of the “T-tube”). Thus, this device comes in the following 3 sizes:

Small: fits PE tubes with inner diameters≦0.87 mm,

Medium: fits PE tubes with inner diameters between 0.88 mm and 1.5 mm,

Large: fits PE tubes with inner diameters>1.5 mm.

A difference between the 3 different sized devices is the inner diameter of the suction tube. Note that the PE tube, when loaded on our device, slides over the suction tube. As with all embodiments discussed throughout, the materials, sizes, dimensions and contours of the device or any components thereof may vary as desired or required for given applications and procedures.

### EXAMPLE 2

### Disposable Device

This exemplary disposable device has same shape and form as the reusable device described above. It also comes in 3 different sizes (or as different as desired). A difference is that it is made out of a cheaper, disposable material (similar to that of disposable needles, or perhaps strong plastic). The disposable device comes pre-packaged in a sterile pack with the PE tube pre-loaded on the tip of the device. The surgeon simply opens the sterile pack, removes the device (containing the PE tube), hooks the distal end of the device into the wall vacuum unit, and places the PE tube into the incision using the device. After the procedure, the disposable insertion device is discarded.

### EXAMPLE 3

### Incorporation of Myringotomy Blade

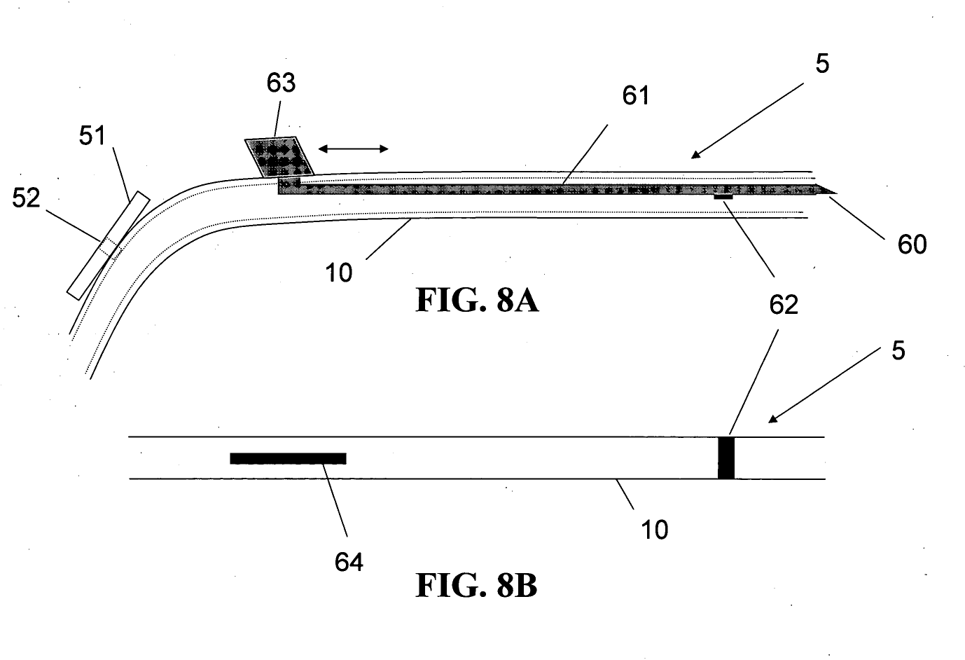

FIG. 8 represents the surgical device 5 incorporating a blade which may be retracted by the user. The cutting blade 60 is mounted on a rod (‘blade rod’) 61. The blade rod runs inside the rod 10, and is supported by the ‘support shelf’ 62. The end of the blade rod is connected to the ‘slide tab’ 63, near the thumb rest 51. The slide tab protrudes through a slit 64 from the rod 10. The slide tab can move forward and backward. When the slide tab is in the forward position, the blade protrudes from the end of the suction tube. When the slide tab is in the backward position, the blade is not exposed. As with any of the embodiments or examples discussed throughout it may be disposable or reusable or combination thereof, as well as pre-packed and/or pre-loaded.

### EXAMPLE 4

### Detachable Collar

This detachable collar acts as a form of adapter, and may take the form of a clear plastic or rubber collar that slides firmly over the existing suction device's tip so that there is a press fit, making the collar stable and preventing it from sliding along the tube or coming loose and sliding off the tube. In this case, the invention is the tight collar that slides over the existing device. An alternative embodiment of this adaptation to the existing suction device is spot welding a burr on the tip of the existing suction device that acts as an insertion collar to push the loaded PE tube into the myringotomy incision.

In summary, as with all embodiments discussed throughout, the materials, sizes, dimensions, shapes and contours of the device or any components thereof may vary as desired or required for given applications and procedures. As with any of the embodiments or examples discussed throughout it may be disposable or reusable or combination thereof, as well as pre-packed and/or pre-loaded.

The composition, devices, systems and methods of various embodiments of the invention disclosed herein may utilize aspects disclosed in the following patents and applications and are hereby incorporated by reference in their entirety:

U.S. Pat. No. 4,468,218 to Armstrong;

U.S. Pat. No. 5,693,065 to Rains, III; and

PCT International Application No. PCT/US06/17906, filed May 5, 2006, entitled “Surgical Tool and Insertion Device for Tube Placement,” of which is hereby incorporated by reference herein in its entirety.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A surgical device in communication with a suction source, said device for suctioning of the middle ear with the insertion and placement of a ventilation tube in the tympanic membrane, said device comprising:
a tubular rod portion having a longitudinal axis, a proximal end, and a distal end with ventilation tube disposed thereon;
an insertion collar portion coaxial to the longitudinal axis of said rod portion to effectively apply a force against the mounted ventilation tube through the tympanic membrane when said rod portion is advanced; and
wherein said rod portion is adapted to allow suction of the middle ear through the distal end of said rod portion during the insertion and/or placement of the ventilation tube.

2. The surgical device, as recited in claim 1, wherein said insertion collar portion is located at a distance from the distal end of said rod portion greater than the axial length of said ventilation tube.

3. The surgical device, as recited in claim 1, wherein said insertion collar portion is located at a distance from the distal end of said rod portion corresponding to the axial length of said ventilation tube.

4. The surgical device, as recited in claim 1, wherein said insertion collar portion is located at a distance from the distal end of said rod portion less than the axial length of said ventilation tube.

5. The surgical device, as recited in claim 1, wherein said collar portion having an extension portion and a non-extension portion, said extension portion having a smaller cross section than non-extension portion thereby defining a shoulder there between;

6. The surgical device, as recited in claim 5, wherein at least a portion of said ventilation tube extends over at least a portion of said extension portion, wherein the applied force is effected by said shoulder contacting said tube.

7. The surgical device, as recited in claim 1, wherein said ventilation tube having a proximal lumen portion and distal lumen portion, said distal lumen portion having a smaller cross section than proximal lumen portion thereby defining a shoulder lumen there between;

8. The surgical device, as recited in claim 7, wherein at least a portion of said ventilation tube extends over at least a portion of said collar portion, wherein the applied force is effected by said shoulder lumen contacting said collar portion.

9. The surgical device, as recited in claim 1, wherein said insertion collar portion is detachable, wherein it can be installed on/or removed from said tubular rod portion.

10. The surgical device, as recited in claim 1, wherein said insertion collar portion and said rod portion are a single integral component.

11. The surgical device, as recited in claim 1, wherein said insertion collar portion and said rod portion are radially axisymmetric.

12. The surgical device, as recited in claim 1, wherein said insertion collar portion is made of a clear material to provide light penetration and/or an unobstructed view of the ear.

13. The surgical device, as recited in claim 1, further comprising a blade.

14. The surgical device, as recited in claim 11, wherein said blade is retractable from said rod portion.

15. The surgical device, as recited in claim 12, wherein said retraction of the blade is controlled by user.

16. The surgical device, as recited in claim 1, wherein said device is made of disposable material.

17. The surgical device, as recited in claim 1, wherein said device is provided in a sterile package or container.

18. The surgical device, as recited in claim 1, wherein said device is pre-loaded with said ventilation tube.

19. The surgical device, as recited in claim 1, wherein said collar portion incorporates one or more protrusions, wherein the applied force is effected by said protrusions contacting said tube.

20. The surgical device, as recited in claim 1, wherein said collar portion extends along said such rod portion, forming an extended sleeve portion.

21. A surgical device in communication with a suction source, said device for suctioning of the middle ear with the insertion and placement of a ventilation tube mounted on a tubular rod portion in the tympanic membrane, wherein said tubular rod portion having a longitudinal axis, a proximal end, and a distal end and is adapted to allow suction of the middle ear through the distal end of said rod portion during the insertion and/or placement of the ventilation tube, said device comprising:
an insertion collar portion coaxial to the longitudinal axis of said rod portion to effectively apply a force against the mounted ventilation tube through the tympanic membrane when said rod portion is advanced.

22. A surgical device for the insertion and placement of a stent in the sinus ostium, said device comprising:
a rod portion having a longitudinal axis, a proximal end, and a distal end with the stent disposed thereon; and
an insertion collar portion coaxial to the longitudinal axis of said rod portion to effectively apply a force against the mounted stent through the sinus ostium when said rod portion is advanced.

23. The surgical device as recited in claim 22, wherein said rod portion is hollow and said surgical device is in communication with a suction source, said device for suctioning of the sinus ostium, and wherein said rod portion is adapted to allow suction of the sinus ostium through the distal end of said rod portion during the insertion and/or placement of the stent.

24. A surgical device for the insertion and placement of a stent in the sinus ostium, wherein said stent is mounted on a rod portion, said device comprising:
an insertion collar portion coaxial to the longitudinal axis of said rod portion to effectively apply a force against the mounted stent through the sinus ostium when said rod portion is advanced.

25. The surgical device as recited in claim 24, where said rod portion is hollow having a longitudinal axis, a proximal end, and a distal end with the stent mounted thereon; and wherein said surgical device is in communication with a suction source, said device for suctioning of the sinus ostium, and wherein said rod portion is adapted to allow suction of the sinus ostium through the distal end of said rod portion during the insertion and/or placement of the stent.

26. The surgical device as recited in claim 22, further comprising a light source or image or video device or combination thereof in communication with said surgical device.

27. The surgical device as recited in claim 24, further comprising a light source or image or video device or combination thereof in communication with said surgical device.
